SEQUENCE LISTING (3 sequences)

SEQ ID NO: 1 - DNA, 19 bp, Artificial Sequence
  Description of Artificial Sequenceprimer
cccactgctgcctcccgta

SEQ ID NO: 2 - DNA, 20 bp, Artificial Sequence
  Description of Artificial Sequenceprimer
tgcacacaggccacaaggga

SEQ ID NO: 3 - DNA, 20 bp, Artificial Sequence
  Description of Artificial Sequenceprimer
agagtttgatcctggctcag

(19) United States
(12) Patent Application Publication
(10) Pub. No.: US 2003/0219778 A1
(43) Pub. Date: Nov. 27, 2003

(54) Method for the diagnosis, identification and characterization of M. tuberculosis and other mycobacteria by shift mobility assay

(75) Inventors: Paulo C.P. Ferreira, Minas Gerais (BR); Erna G. Kroon, Minas Gerais (BR); Maria E.B.M. Pinto, Minas Gerais (BR); Agdemir W. Aleixo, Minas Gerais (BR)

Correspondence Address: NIXON & VANDERHYE, PC, 1100 N GLEBE ROAD, 8TH FLOOR, ARLINGTON, VA, 22201-4714, US

(73) Assignee: UNIVERSIDADE FEDERAL DE MINAS GERAIS - UFMG

(21) Appl. No.: 10/330,359

(22) Filed: Dec. 30, 2002

Related U.S. Application Data
(63) Continuation of application No. 09/582,661, filed on Aug. 29, 2000, now abandoned.
Application No. 09/582,661 is a 371 of international application No. PCT/BR97/00087, filed on Dec. 30, 1997.

(51) Int. Cl. C12Q 1/68; C12P 19/34

(52) U.S. Cl. 435/6; 435/91.2

(57) ABSTRACT

The present invention relates to a method for diagnosis, identification and characterization of M. tuberculosis or any other mycobacteria by using PCR and shift mobility assay (SMA). The method is based both on microheterogeneities observed within the 16S rRNA sequences established for mycobacteria and on nucleotide gaps and mismatches which cause a decrease in the electrophoretic mobility of DNA heteroduplex in polyacrylamide gels. The PCR strategy is based on the use of a specific primers for mycobacteria genera and divergence in sequences found in 16S rRNA coding gene. The difference in nucleotide sequence was enough to identify mycobacteria species, since a remarkable shift between single stranded and homoduplex bands in PAGE were observed among mycobacteria tested, when Mycobacterium tuberculosis was used as standard. The shift of PCR product of bacteria other than mycobacteria was observed above the single stranded band in PAGE, both in standard cultures and clinical specimens. SMA can thus provide a fast and sensitive method for detection and classification of mycobacteria in clinical samples as well as pure culture.

### TECHNICAL FIELD OF THE INVENTION

The present invention relates to a method for detect, identify and classify Mycobacterium tuberculosis or any other mycobacteria, by using a urea-poliacrylamide gel (UPAGE) to distinguish between heteroduplex and homoduplex shift bands obtained by mixing PCR products derived from 16S rRNA coding gene fragment of mycobacteria. The method is based on divergence in sequences found in 16S rRNA to identify mycobacteria species, since a remarkable shift of heteroduplex bands are obtained between single stranded and homoduplex bands in UPAGE.

### BACKGROUND OF THE INVENTION

Tuberculosis (TB) and other mycobacterial illnesses remain the number one cause of infectious diseases related deaths worldwide. Annual increases of 10% per year have been reported (Bloom, B. R., and C. J. Murray. Science 257:1055-1064, 1992; Centers for Disease Control. MMWR 40:58, 1991) in part related to the epidemic of HIV. At the same time an increasing number of atypical presentations of AIDS patients with Mycobacterium tuberculosis (MTB) are also seen, with a vast increase in extra pulmonary diseases (Persing, D. H., J. Clin. Microbiol. 29:1281-1285, 1991). Of even greater concern than the increase in absolute numbers of mycobacterial infections is the emergence of previously rare strains, collectively termed mycobacteria other than tuberculosis (MOTT). Moreover, of particular importance, is the emergence of MTB multi drug-resistant, which causes over 90% mortality in immunocompromised hosts (Shankar, P., N. Manjunath, R. Lakshmi, B. Aditi, P. Seth, and Shriniwas. Lancet 335:423, 42. 1990).

The conventional diagnosis of tuberculosis based on the gold standard, culture-grown organisms, is extremely sensitive (10-100 organisms per ml present for growth) but is expensive and time-consuming (2-8 weeks), because of the slow doubling rate (Vaneechoutte M., H. Beenhouwer, C. Claeys, G. Verschraegen. A. de Rouck, N. paepe, A. Alachouni, and F. Portaels. J. Clin. Microbiol. 31:2061-2065. 1993) of acid-fast bacilli (AFB). Direct staining procedures are rapid but lack sensitivity and specificity since Legionella and Nocardia are stained as well (Rogall, T., J. Wolters, T. Flohr, and E. C. Boettger. Int. J. Syst. Bacteriol. 40:323-330, 1990).

By overcoming the inherent limitations of stain and culture techniques, molecular methods have great potential for the rapid diagnosis of mycobacteria. The polymerase chain reaction (PCR) was reported (Cousins, D. V., S. D. Wilton, B. R. Francis, and B. L. Gow. J. Clin. Microbiol. 30:255-258, 1992) to be useful in the direct diagnosis of tuberculosis infection directly from a variety of clinical specimens (Bocart, D., D. Lecossier, A. Lassence, D. Valeyre, J. P. Battesti, and A. J. Hance. Am Rev Respir Dis 145:1142-1148,1992; Brisson-Noèl, A., B. Gicquel, D. Lecossier, V. Lévy-Frebault, X. Nassif, and A. J. Hance. Lancet ii:1069-1071, 1989) with a diversity of genetic elements used as target templates. The rapid detection of MTB by PCR and the high degree of sensitivity in clinical samples afforded by DNA amplification have been described by various investigators (Cousins, D. V., S. D. Wilton, B. R. Francis, and B. L. Gow. J. Clin. Microbiol. 30:255-258, 1992 Del Portillo, P., L. A. Murillo, and M. E. Patarroyo. J. Clin. Microbiol. 29:2163-2168, 1991; Folgueira, L., R. Delgado, E. Palenque, and A. R. Noriega. J. Clin. Microbiol. 31:1019-1021, 1993). However, there are a number of difficulties to be overcome before these methods can be used as a routine diagnosi test (Garcia-de-Lomas J. and D. Navarro. J. 16S43-S48, 1997). Nevertheless, PCR analysis of chromosomal DNA is a simple and rapid method for identifying MTB to the species level and in clinical specimens (Shawar, R. M., F. A. K. El Zaatari, A. Nataraj, and J. E. Clarridge. J. Clin. Microbiol. 31:61-65, 1993).

PCR reaction probes and primers for the insertion fragment IS986 or IS6110 (Cave, M. D., K. D. Eisenach, P. F. MacDermot, J. H. Bates, and J. T. Crawford. Mol. Cel. Probes 5:73-80, 1991; Chevrel-Dellagi, D., A. Abderrahman, R. Haltiti, H. Koubaji, B. Gicquel, and K. Dellagi. J. Clin. Microbiol. 31:2446-2450, 1993) have been used and many different strategies have been proposed to confirm the identity of amplified DNA products. One common strategy for detecting and speciating microorganisms is specific gene-probe hybridization and PCR targeted to 16S rRNA subunit (Bõddinghaus, B., T. Rogall, T. Flohr, H. Blõcker, and E. C. B\overline{o}ttger. J Clin Microbiol 28:1751-1759, 1990), which is coded in mycobacteria by multiple gene (10 genes for 16S RNA) copies per cell. The 16S subunit contains sequences that have highly conserved regions virtually identical in all bacteria (FIG. 7), interspersed with other sequences found to be genus- and/or species-specific (Victor, T., R. DuToit, and P. D. VanHeiden. J. Clin. Microbiol. 30:1514-1517, 1992). Conserved regions can be used to amplify rRNA from all prokaryotes or from broadly-related groups of bacteria, while the genus and species-specific interspersed sequence domains can be used to construct specific oligonucleotide probes to detect and differentiate resulting PCR products, or to directly probe mycobacteria in clinical samples by hybridization.

Most often, PCR products are visualized with ethidium bromide staining on an agarose gel (FIG. 1), with final confirmation on Southern blot. Previously, colorimetric PCR hybridization assays have been investigated as an alternative to gel electrophoresis and Southern blot analysis of PCR-amplified DNA. Such gels and Southern blots have proven to be time-consuming and tedious for typical clinical laboratories as well. Solid-phase colorimetric PCR assays capture denatured amplification products on probes bound to nylon membranes (as in reverse dot blots) (Plikaytis, B. B., K. D. Eisenach, J. T. Crawford, and T. M. Shinnick. Molec. Cell. Probes 15:215-9, 1991; Sambrook, K. J., Fritsch, E. F., Maniatis, T., In: Molecular Cloning. A Laboratory Manual USA: Cold Spring Harbor Laboratory Press,1989) or to microtiter plates (Thierry, D., M. D. Cave, K. D. Eisenach, J. T. Crawford, J. H. Bates, B. Gicquel, and J. L. Guesdon. Nucleic Acids Res. 18:188-189, 1990). However, the sensitivity of these assays suffer due to the tendency of the denatured PCR product strands to reassociate and exclude oligonucleotide probes, and stearic interference between the bound oligonucleotides and the solid support which impede hybridization to nucleic acids in solution (Plikaytis, B. B., K. D. Eisenach, J. T. Crawford, and T. M. Shinnick. Molec. Cell. Probes 15:215-9, 1991).

Other difficulties encountered using existing techniques include variability from sample preparation and detection of PCR products (Nolte, F. S., B. Metchock, J. E. McGowan, A. Edwards, O. Okwumabaa, C. Thurmond, P. S. Mitchell, B. Pilkaytis, and T. Shinnick. J. Clin. Microbiol. 31:1772-1782, 1993), technically unmanageable steps to enhance detection, false-positive reactions due to the amplification of contaminating DNA and inhibition of DNA amplification (Noordhoek, G. T., A. H. J. Kolk, G. Bjune, D. Catty, J. W. Dale, P. E. M. Fine, P. Godfrey-Fausset, S-N. Cho, T. Shiinick, S. B. Svenson, S. Wilson, and J. D. A. van Embden. J Clin Pathol. 48: 810-914, 1995). These difficulties argue for searching more precise, sensitive, reproducible and low cost assays for MTB diagnosis.

We report here a method for detection and identification of mycobacteria using PCR strategies tare to mycobacterial 16S rRNA coding gene and the heteroduplex mobility assay previously developed for analysis of HIV (Delwart, E. L., Herring, B., Rodrigo, A. G. and Mullins, J. I. PCR Meth. and Appl., 1995; Delwart, E. L., Shepaer, E. G., Louwagie, J., McCutchan, F. E. Grez, M., Waigmann, H. R., and Mullins, J. I. Science 262:1257-1261, 1993). This new approach is fast, simple and can produce information not easily obtained when compared with other methods described so far. Thus, combining molecular methods and PAGE we developed a method which resulted in an integrated strategy to achieve sensitive and specific diagnosis of mycobacteria. Combination of these approaches has wide application in detecting microorganisms and in detection of gene products important to clinical genetic and neoplastic disease.

### BRIEF DESCRIPTION OF THE DRAWINGS

These and other objects, features and many attendant advantages of the invention will be better understood upon a reading of the following detailed description when considered in connection with the accompanying drawings wherein:

FIG. 1 shows a photography of ethidium bromide-stained of PCR amplification products with primers F-285 e ZR-244 derived from DNA of patients with M. tuberculosis and E. coli standard culture. The PCR products (5 μl) were fractioned on a 1% gel agarose at 100 volts for 40 min. and showed an expected 360 bp fragment. Lane M, PEL phage DNA digest with Hind III; lane 2, M. tuberculosis; lane 3, no DNA template; lane 4 to 13 show fragments amplified from clinical samples DNA.

FIG. 2 shows a photography of a shift mobility assay of 360 bp PCR fragment product derived from 16S rRNA coding gene of various bacteria using as standard M. tuberculosis. The PCR product fragment of various bacteria were mix (2.5 μl v/v) with mycobacteria, denatured (95° C.), re-annealed and run in PAGE at 100 volts for 60 min. Lane 1, Proteus mirabilis; lane 2, Proteus mirabilis+M. tuberculosis; lane 3, Proteus mirabilis+M. avium; lane 4, Proteus mirabilis+M. fortuitum; lane 5, Pseudomonas aeruginosa; lane 6, Pseudomonas aeruginosa+M. tuberculosis; lane 7, Pseudomonas aeruginnosa+M. Avium; lane 8, Pseudomonas aeruginosa+M. fortuitum; lane 9, Klebisiela sp; lane 10, Klebisiela+M. tuberculosis; lane 11, Klebisiela+M. avium; lane 12. Klebisiela sp+M. fortuitum.

FIG. 3 shows a photography of a shift mobility assay of 1030 pb PCR fragment derived from 16S rRNA coding gen of Mycobacteria. A PCR fragments from M. tuberculosis (standard) and various mycobacteria were mixed (2.5 μl v/v). After denaturation (95° C.). re-annealed reactions were run in PAGE at 200 volts for 60 min. Lane 1, M. tuberculosis; lane 2, M. tuberculosis+M. avium; lane 3, M. avium; lane 4, M. tuberculosis+M. fortuitum; lane 5, M. fortuitum; lane 6, M. tuberculosis+M. smegmatis; lane 7, M. smegmatis; lane 8, M. tuberculosis+M. kansasii; lane 9, M. Kansasii; lane 10, M. tuberculosis+M. scrofulaceum; lane 11. M. scrofulaceum.

FIG. 4 shows a photography of effect of M. fortuitum derived 16S rRNA 1030 pb PCR product concentration in the shift mobility assay detection. A PCR fragments from standard M. tuberculosis (1460 ηg) and M. fortuitum were mix (2.5 μl v/v), and denatured (95° C.). After re-annealed reactions were run in 5% PAGE with 3% of urea in TBE buffer at 200 volts for 60 min. Lane 1. M. tuberculosis; lane 2, M. fortuitum (1500 ηg); lane 3, M. tuberculosis+M. fortuitum (1500 ηg); lane 4, M. tuberculosis+M. fortuitum (800 ηg), lane 5, M. tuberculosis+M. fortuitum. (400 ηg); lane 6, M. tuberculosis+M. fortuitum. (200 ηg).

FIG. 5 shows a photography of effect of DNA template concentration in the. heteroduplex mobility assay of 1030 pb PCR fragment derived from rDNA of M. tuberculosis e M. fortuitum. A PCR fragments from M. tuberculosis (standard) and M. fortuitum derived from different DNA template concentration were mixed (2.5 μl v/v) denatured (95° C.). re-annealed and run in PAGE at 200 volts for 60 min. Lane 1, M. tuberculosis, (100 ηg); lane 2, M. fortuitum. (100 ηg); lane 3, M. tuberculosis (10 ηg)+M. fortuitum. (10 ηg); lane 4, M. tuberculosis (5 ηg)+M. fortuitum (5 ηg); lane 5, M. tuberculosis (1 ηg)+M. fortuitum (1 ηg); lane 6, M. tuberculosis (0.1 ηg)+M. fortuitum (0.1 ηg); lane 7, M. tuberculosis (0.01 ηg)+M. fortuitum. (0.01 ηg).

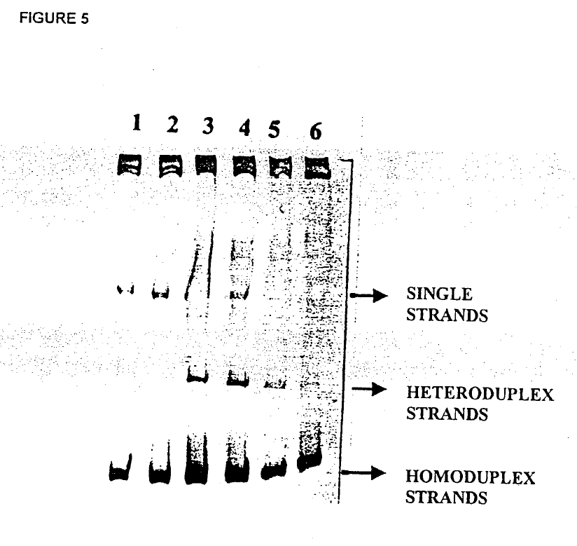

FIG. 6 shows the identification of M. avium by heteroduplex mobility shift assay in clinical sample of patient suspected of mycobacteria infection. The assay was peformed by using M. tuberculosis and M. fortuitum as a standard. A PCR fragments from M. tuberculosis or M. fortuitum were mix (2.5 μl v/v) to clinical sample #1 and #2 denatured (95° C.) re-annealed and run in PAGE at 200 volts for 60 min. Clinical sample #1 showed in lanes 1 to 4 is a typical profile pathem of M. tuberculosis and and clinical sample #2 (lanes 5 to 8) of M. avium. Lane 1, sample clinical #1; lane 2, sample clinical #1+M. tuberculosis; lane , sample clinical #1+M. avium; lane 4, sample clinical #1+M. fortuitum; lane 5, sample clinical #2; lane 6, sample clinical #2+M. tuberculosis, lane, sample clinical #2+M. avium lane 8, sample clinical #2+M. fortuitum.

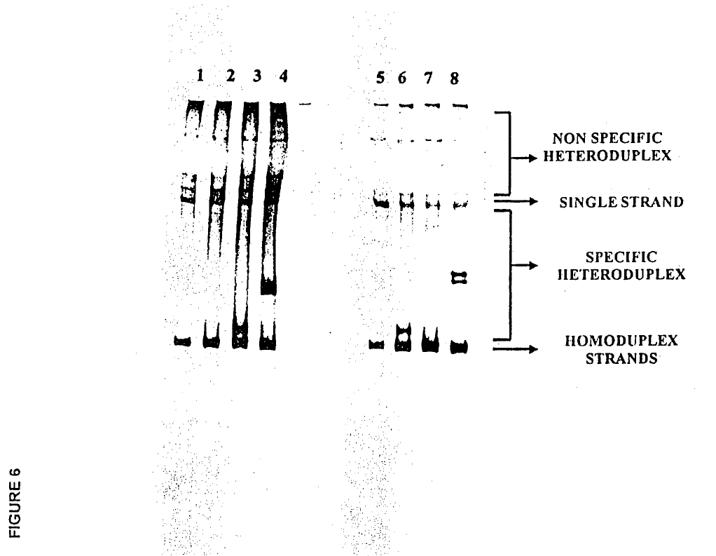

FIG. 7 shows a diagram of 16S RNA and regions where nucleotide sequences are different between some mycobacteria species and E. coli.

### DETAILED DESCRIPTION OF THE INVENTION

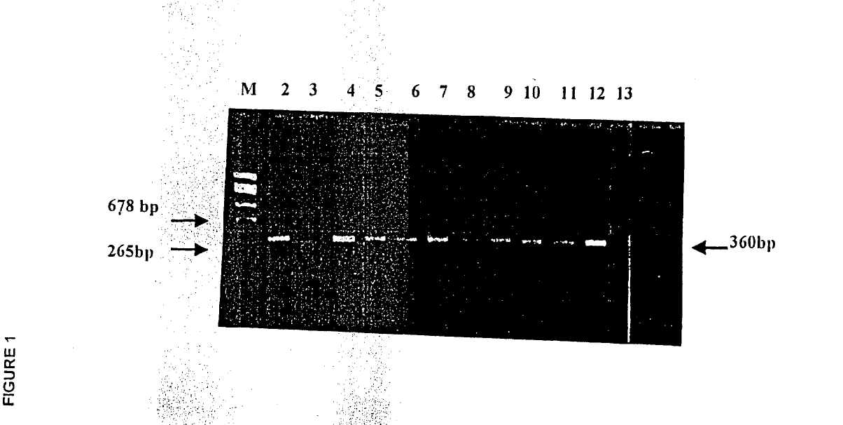

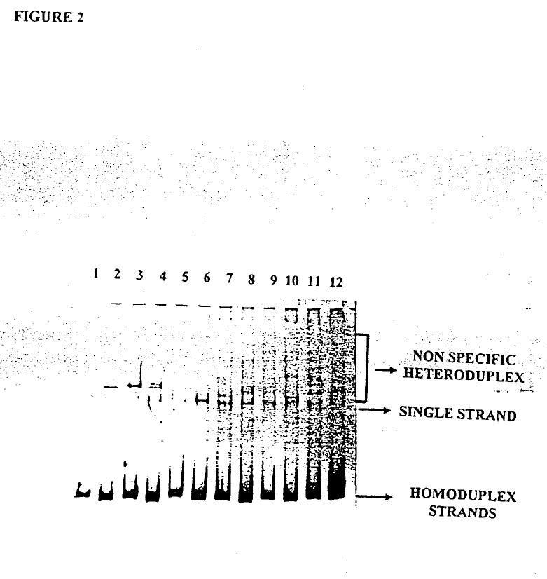

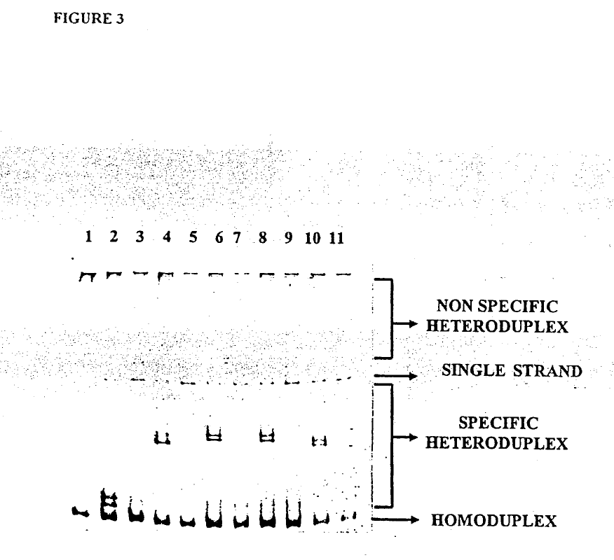

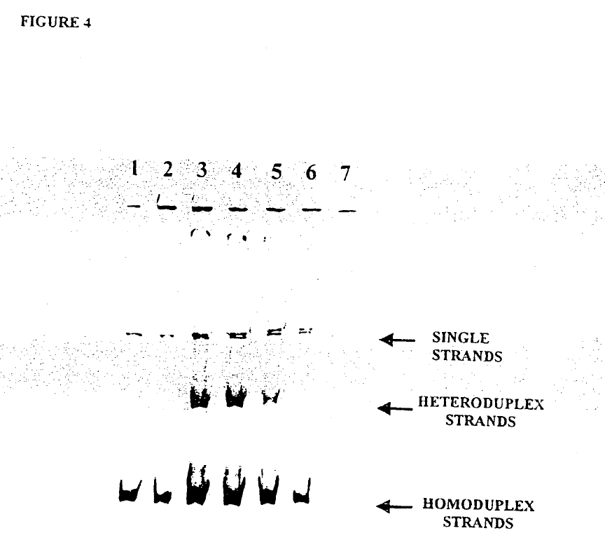

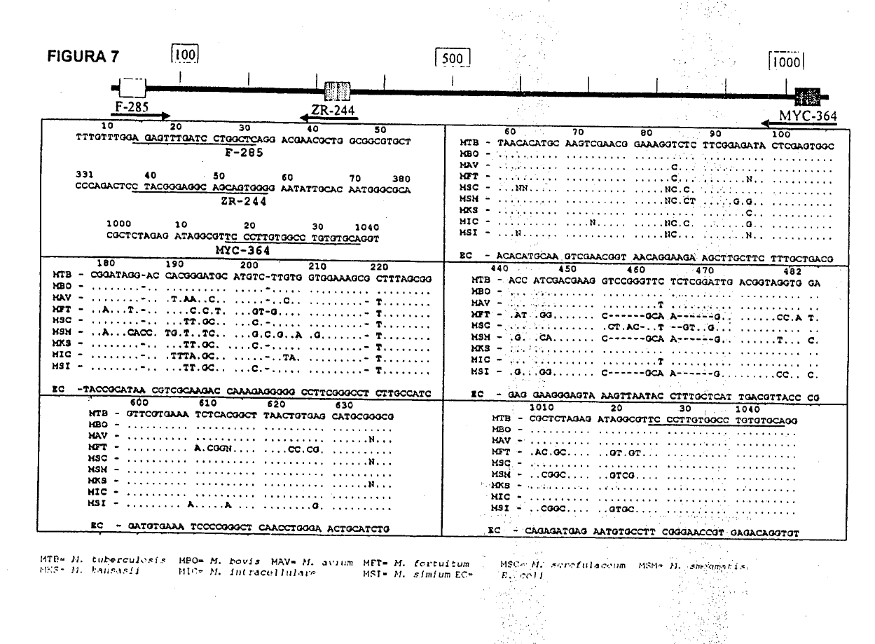

It is, therefore, an object of the present invention to provide a method of identification, classification or diagnosis for Mycobacterium tuberculosis or other mycobacteria that uses the Shift Mobility Assay (SMA). The method outlined here allowed identification of mycobacterium species from culture media or clinical samples based on heteroduplexes formed from 16S, rRNA coding genes. The method is based on heteroduplexes migration in polyacrylamide gels observed after denaturing and reannealing mixtures of PCR products derived from various sources of 16S rRNA genes using M. tuberculosis or M. bovis as standard (FIG. 1) When two non divergent sequence fragments were mixed two bands were observed in polyacrylamide gel: one in the bottom (homoduplex) and other in the middle of the gel, which corresponds to the single stranded DNA (FIG. 2) due to minor differences in primer concentration, heteroduplexes with reduced mobility are detected when annealed DNA has 1-2% divergent nucleotide sequences or when gaps were presented in one relative to a second sequence (FIG. 7). Heteroduplexes were found above the single stranded DNA bands (close to the top of the gel) and between the homoduplexes (bottom of the gel) and single stranded bands. The HMA of amplified PCR products from mycobacteria rDNA derived from culture is shown in FIG. 2 and 4. Specific heteroduplexes were formed between species, even when more than two species were presented in the mixture. Heteroduplex formation was also observed by mixing mycobacteria and other species of bacteria (FIG. 3), but these heteroduplexes migrated slower than the single stranded DNA. Faster migrating heteroduplexes with mobilities faster than ssDNA were observed among all mycobacteria species compared (FIG. 4, 5 and 6).

A variety of commercially available primers in the 16S rRNA coding gene region may be used for PCR amplification.

In order that this invention may be better understood the follow examples for illustrative purposes only, are described. The examples ilustrate the present invention and are not intended to limit it in spirit or scope.

### EXAMPLE 1

Bacterial cultures. Bacterial strains as Mycobacterium bovis, Mycobacterium avium; Mycobacterium scrofulaceum; Mycobacterium kansasii; Mycobacterium smegmatis; Mycobacterium fortuitum or other than mycobacteria (Pseudomonas aeruginosa; Staphylococcus sp.; E. coli; Klebsiela sp. e Proteus mirabilis) obtained from the Lowestein's, agar plate or liquid culture media

### EXAMPLE 2

DNA extraction DNA was extracted after lysing 0.1-1000 μg of solid microorganism from pure culture with 100-500 μl of lysis buffer solution (TE pH 6.0-8.0, 0.1-2.0% of lysozyme, 0.1-10% of Tween 80) for 30-60 min. at 15-30° C. SDS was then added to 1-10% plus 50-500μg/ml of proteinase K and the test tube kept for 40-120 min. at 37-55° C. From clinical samples, aliquots of fresh sputum were treated with N-acetyl, L-cysteine (0.5-2.0 μg/ml) for 40-60 min, at room temperature, followed by treatment with same volume of lysis buffer as described above. DNA was extracted twice with phenol:chloroform: isoamylic alcohol as described by Boddinghaus et al. (Bõddinghaus, B., T. Rogall, T. Flohr, H. Blõcker, and E. C. B\overline{o}ttger. J. Clin Microbiol 28:1751-1759, 1990) ressuspended in deionized H₂O or any other hydrophylic solvent and storage at −20 to −80° C. until use. DNA was measured by spectrophotometry.

### EXAMPLE3

DNA amplification. DNA was amplified by using three oligonucleotides primers inside 16S rRNA of M. tuberculosis were used: two reverse designated primer ZR-244 (CCCACTGCTGCCTCCCGTA) located at nucleotides 298 to 317, MYC-264 located at positions 1027 to 1046 (TGCACACAGGCCACAAGGGA) and a foward primer designated F-285 (AGAGTTTGATCCTGGCTCAG) corresponding to position 8 to 28. The resulting PCR product was 1030 bp long when primers F-285 and MYC-264 were used and 360 bp long for F-285 and ZR-244. PCR reaction containing 0.1-900 μg of template DNA as indicated, 2.0-6.0 mM of MgCl₂, 4-6 pM of each primer in 50-150 mM Trisma (Sigma, SP, Brazil), pH 7.2-8.3, 50 to 200 μM of each dNTP (Pharmacia, São Paulo, Brazil), 2.5-5.0 units of Taq DNA polymerase (Promega, Belo Horizonte, MG, Brasil) and the reaction was performed in a final volume of 20-100 μl. PCR reactions were carried out in a Perkin Elmer 4800 thermal cycler (Perkin Elmer/Cetus) for 35-40 cycles, using one sec ramp setting time between steps in a cycle of 93-96° C. for 1-3 min., 50-65° C. for 1-5 min., 70-72° C. for 1-5 min. and a 10-20 min. 65-72° C. extension step was linked to the last cycle. The annealing temperature for primers F-285 and MYC-264 was 45-70° C. instead of 37-50° C. used for primers F-285 and ZR-244. Positive t,0090 controls were 0.3 μg of DNA from M. bovis (BCG). Two negative controls were included: one with no DNA template and the other with E coli DNA (0.5-2 μg) in the PCR reaction and no amplification products were observed in these reactions PCR products were evaluated by electrophoresis on 2-6% agarose gels and stained with ethidium bromide.

### EXAMPLE 4

Shift mobility assay. The shift mobility assay was performed by mixing 2.5-10 μt of PCR products derived from standard M. tuberculosis PCR product with 2.5-10 μl of PCR product from other species or clinical samples plus 1 μl of 10×annealing buffer (1-5 mM NaCl, 100-500 mM Tris-Cl pH7.2-8.0 and 20-40 mM EDTA). The mixture was heated 90-96° C. for 2-5 min. then cooled to 25-45° C. in the thermocycler. Heteroduplexes were electrophoresed on 3-6% polyacrylamide gels (25-30:0.8-1.0 Acrylamide:Bis) and 3-7% of urea Gels were run at 100-200 volts when the 1030 bp fragment PCR products were used and at 50-100 volts for 360 bp fragments, both for 1 hour, 1×TBE buffer in a mini gel (Hoeffer mini-gel SE 200) apparatus. Gels were than fixed and stained with silver (Stain Plus, Bio-rad, USA).

While the present invention has been described in connection with examples, it will be understood that modifications and variations apparent to those ordinary skill in the art are within the scope of tie present invention.

What is claimed is:

1. A method for diagnosis identification and characterization of M. tuberculosis or any other mycobacteria by using PCR and shift mobility assay (SMA) in urea-poliacrylamide gel electrophoresis, comprising the following steps: 1) culture: 2) DNA extraction: 3) DNA amplification: 4) Shift mobility assay

2. The method according claim 1, wherein the the step 1 of culture can optional

3. The method according claim 1, wherein the DNA extraction (2) from culture (1) or clinical samples can be made with phenol/chlorophorm extraction method or any other method

4. The method according claim 1, wherein the DNA amplification can be made by using any primer in the region of 16S rRNA coding gene, or using any other gene than 16S rRNA coding gene

5. The method according claim 1, wherein the shift mobility assay are made use of urea in poliacrylamide gel electrophoresis to detect, visualize or identify by PCR products derived from sequences of 16S ribossomal RNA coding gene and for detection of heteroduplex complex observed between single strand (middle of migration) and homoduplex complex bands (bottom of the gel);

6. The method according claim 1, wherein use of M. tuberculosis or any other mycobacteria as standard to identify by shift mobility assay PCR products derived from sequences of 16S ribossomal RNA coding gene or any other gene.
